(19) United States
(12) Patent Application Publication
(10) Pub. No.: US 2024/0382627 A1
(43) Pub. Date: Nov. 21, 2024

(54) NON-METALLIC TEMPERATURE-RESPONSIVE COMPOSITE FOR MAGNETIC RESONANCE IMAGING (MRI), AND PREPARATION METHOD AND USE THEREOF

(71) Applicant: SOUTH CHINA UNIVERSITY OF TECHNOLOGY, Guangzhou (CN)

(72) Inventors: Linge WANG, Guangzhou (CN); Yifan JIA, Guangzhou (CN); Qianqian YU, Guangzhou (CN)

(73) Assignee: SOUTH CHINA UNIVERSITY OF TECHNOLOGY, Guangzhou (CN)

(21) Appl. No.: 18/273,045

(22) PCT Filed: Sep. 27, 2022
(86) PCT No.: PCT/CN2022/121837

§ 371 Date: Jul. 19, 2023

(30) Foreign Application Priority Data
Mar. 30, 2022 (CN) 202210326274.4

(51) Int. Cl. A61K 49/12

(52) U.S. Cl. CPC A61K 49/12; A61K 2123/00

(57) ABSTRACT

The present disclosure belongs to the field of functional fiber materials, and discloses a non-metallic temperature-responsive composite for magnetic resonance imaging (MRI), and a preparation method and use thereof. An organic hydrogen-containing molecular contrast agent is loaded into a polymer fiber through electrospinning, and the phase structure and molecular motility of the organic hydrogen-containing molecular contrast agent are controlled to regulate its relaxation time. The non-metallic temperature-responsive composite can achieve an “on/off” temperature-responsive MRI effect for a polymer material without the addition of an additional magnetic field. Compared with the prior art, an additional magnetic field is not required; the selected contrast agent is non-toxic or exhibits low toxicity to the human body; and an “on/off” imaging effect can be realized, where the advantages of nuclear magnetic resonance (NMR) signals of the material itself will not be affected when imaging is not conducted.

### TECHNICAL FIELD

The present disclosure belongs to the field of functional fiber materials and specifically relates to a non-metallic temperature-responsive composite for magnetic resonance imaging (MRI) and a preparation method and use thereof.

### BACKGROUND

### MRI

Magnetic resonance (MR) refers to a physical phenomenon that an atomic nucleus resonates with an applied magnetic field under specified conditions. A basic working principle of magnetic resonance imaging (MRI) is as follows: An object to be tested is placed in a special magnetic field, and a radio frequency (RF) pulse is used to excite hydrogen nuclei in the object to make hydrogen nuclei resonate and absorb energy. After the RF pulse is stopped, hydrogen nuclei release the absorbed energy and emit a radio signal at a specific frequency, and the radio signal is acquired by a receiver of an MRI device and processed by a computer to obtain MR image. At present, primary signal source of MRI device is hydrogen atom. (see Yang Zhenghan et al., “Guide to Technique of Magnetic Resonance Imaging”, People's Military Medical Press, 2010: P18-19).

### Signal Intensity and Relaxation Time of MRI

After being excited by an RF pulse, hydrogen atoms in different states (such as chemically bonded, filled in pores, and in different phase structures) release energy at different rates when returning to a ground state. This energy-release process is called relaxation. There are two separate relaxation processes, which are called T1 relaxation (or longitudinal relaxation) and T2 relaxation (or transverse relaxation); the times consumed for these two relaxation processes to occur are called T1 relaxation time and T2 relaxation time.

MRI has a variety of scanning sequences for the acquisition of signals, where the most common sequences are T1WI (T1-weighted imaging), T2WI (T2-weighted imaging), PDWI (proton density-weighted imaging), and DWI (diffusion-weighted imaging). Signal intensities of images acquired by these common scanning sequences are affected by the T1 relaxation time and the T2 relaxation time of hydrogen atoms, namely, hydrogen atoms with varying relaxation times contributes different signal intensities on MR images (see Ray H. Hashemi et al. MRI: The Basics. Lippincott Williams & Wilkins, 2012, p54-55). s For T1WI, T2WI, and PDWI scanning sequences, the relationship between MRI signal intensity and relaxation times is as follows:

$$S ∝ {\sum }_{i = 1}^{∞} {N}_{( H ) i} * ( 1 - {e}^{- \frac{TR}{T 1}} ) * {e}^{- \frac{TB}{T 2}} \qquad formula ( 1 )$$

where S represents an MRI signal intensity; N_{(H)i} represents a number of hydrogen atoms with the T1 relaxation time and the T2 relaxation time; T1 and T2 represent the T1 relaxation time and T2 relaxation time of a hydrogen atom, respectively; TR and TE represent a repetition time and an echo time, respectively. TR and TE are components of a scanning sequence, and their values are designed for a human tissue; the summation symbol indicates that all hydrogen atoms in the area provide MRI signals for an image.

Polymer materials cannot produce enough MRI signals.

Polymer materials have been widely used in the manufacture of medical devices, but hydrogen atoms in polymer materials have a short T2 relaxation time, which is in a range from tens of milliseconds to tens of nanoseconds. For an MRI device, a minimum value of TE in formula (1) is a few milliseconds, and it means that for a substance with a T2 relaxation time of only 1 ms, if a scanning parameter TE is 5 ms, a value of this parameter alone is

$${e}^{- \frac{5 ms}{1 ms}} = 6.74 * {10}^{- 3} .$$

This value is several orders of magnitude lower than an MRI signal intensity provided by a water molecule. Due to a limitation of the MRI device itself, it is difficult to detect a hydrogen atom with such a short relaxation time, such that a polymer cannot be imaged by MRI in vivo. This phenomenon is described in the study of Yuan et al. (Yuan D C et al. Journal of Biomedical Materials Research Part B-Applied Biomaterials, 2019, 107 (7): 2305-2316), where the polymer (polybutylene succinate (PBS)-butylene terephthalate) fiber is used as an artificial intervertebral disc instead of a nucleus pulposus, but it is completely signal-free under MRI. This problem makes it difficult for a doctor to acquire information about an implanted polymer material in a patient through MRI, which hinders treatment.

Disclosed temperature-responsive imaging techniques and defects thereof.

There are a variety of techniques for producing MRI contrast, where one common method is using contrast agents. Delivering magnetic substance to the target area through injection or oral administration causes, and the magnetism carried by this substance will shorten the T1 and T2 relaxation times of nearby hydrogen atoms (usually derived from water molecules). Such a substance is called a contrast agent. The presence of a contrast agent will cause the enhancement of an MRI signal intensity on a T1WI sequence and the reduction of MRI signal intensity on a T2WI sequence, and such signal changes will lead to a high signal contrast between a target area and surrounding environment, thereby achieving an imaging contrast. An imaging efficiency of a contrast agent can be described by the physical quantity relaxivity:

$$\frac{1}{{T}_{i}} = {R}_{i} = {R}_{0} + {r}_{i} \cdot [ CA ] \qquad formula ( 2 )$$

where i=1 or 2, which represents T1 or T2 relaxation; Rᵢ represents a relaxation rate, which is a reciprocal of a relaxation time Tᵢ with a unit of s⁻¹; [CA] represents a concentration of a contrast agent with a customary unit of mmol/L; and rᵢ represents a relaxivity with a customary unit of L/(mmol·s).

The formula (2) shows that the addition of a contrast agent will cause a change in the relaxation rate; a relaxation rate has a linear relationship with a concentration of a contrast agent; and a slope represents relaxivity. The relaxivity indicates a change in the relaxation rate per unit concentration of a contrast agent, which means that, a contrast agent with a higher relaxivity leads to a stronger contrast effect when used at the same concentration.

The relaxivity of a contrast agent is affected by many factors, including size, shape, surface modification, chelate structure, etc. For the disclosed temperature-responsive contrast agents, temperature changes are designed to change the above properties of contrast agents. When a temperature changes, a contrast agent transitions between high relaxivity and a low relaxivity state (see Hingorani D V et al. Contrast Media & Molecular Imaging, 2014, 10 (4): 245-265). A temperature-responsive contrast agent can be loaded into a polymer material, such that, when a temperature changes, the relaxivity of the contrast agent for water molecules around the polymer material will also change between a high state and a low state, thereby reflecting a temperature distribution in vivo.

However, the technical solutions disclosed above have the following problems: (1) The disclosed temperature-responsive contrast agent require the generation of an additional magnetic field in target area (the magnetism is usually provided by a metal atom), and the injection of such contrast agents into the human body can cause uncomfortable reactions such as an allergy (see Semelka R C et al. Magnetic Resonance Imaging, 2016, 34 (10): 1399-1401 and Daldruplink H E. Radiology, 2017, 284 (3): 616-629). (2) The disclosed temperature-responsive contrast agents can transition between a high relaxivity state and a low relaxivity state as temperature changes, but an “on” imaging effect and an “off” imaging effect cannot be provided, and thus an area in which the contrast agent is located is always in a contrast enhanced state. If such a contrast agent is loaded within a polymer material, the polymer material will always be in a contrast enhanced state, and even in a low relaxivity state of the contrast agent, The signal where polymer material locates will be affected by the contrast agent. As a result, signals generated by substances such as cells and liquids that migrate to the internal space inside the polymer material are also altered by the contrast agent. No matter which relaxivity state the contrast agent is in, the MRI signal intensity cannot directly reflect a real state of substances such as cells inside the polymer material, which limits the application of the contrast agent in tissue engineering scaffolds and other fields.

### SUMMARY

In view of the defects in the prior art, an objective of the present disclosure is: (1) to provide a method for MRI using a non-metallic temperature-responsive composite and illustrate a principle thereof; (2) to provide a preparation method of a non-metallic temperature-responsive composite for MRI and process parameters thereof; and (3) a use of the composite. (a) The present disclosure can achieve temperature-responsive MRI of a polymer material without the introduction of an additional magnetic field. (b) The temperature-responsive imaging refers to an “on/off” imaging effect, rather than the transition between a high relaxivity and a low relaxivity, such that, when the imaging effect is “off”, a signal of a position of the material is totally derived from an environment in which the polymer material is located (including an infiltrated body fluid, a proliferated cell, or the like) without being interfered by the contrast agent.

A principle of the present disclosure to achieve the above effect is as follows:

The present disclosure provides a method for regulating relaxation times of such a compound through phase structure transition, and a principle of regulating a relaxation time through phase structure transition is as follows: a relaxation time of a hydrogen atom is affected by a variety of relaxation mechanisms, and hydrogen atoms in different phase structures undergo different primary relaxation mechanisms. Therefore, substances with different phase structures have different relaxation times. FIG. 1 shows a relationship between relaxation times (T1, T2) of an atom and a correlation time (τ_{c}), where τ_{c} is inversely proportional to a molecular motility. It can be seen from FIG. 1 that, due to strong motility, liquid molecules have small τ_{c} and long T1 and T2 relaxation times; and due to large molecular sizes, polymer molecules and protein molecules have weakened motility and increased τ_{c}. Although many polymers are in a solid state at room temperature, some polymers (such as silica gel) have an extremely low glass transition temperature and molecular chains thereof can still move freely at room temperature, such that these polymers have shorter relaxation times than liquid molecules. Due to the production of a crystal, crystalline molecules are confined to lattice sites, cannot move freely, and can only vibrate in situ, such that τ_{c} is large. When molecules constituting a substance change from a freely-moving liquid phase and an amorphous state to a crystalline state confined in lattices, T2 relaxation time of the substance can be reduced by several orders of magnitude. According to formula (1), such a large change in T2 relaxation time can lead to a large change in MRI signal intensity. Therefore, when a substance transitions between a freely-moving liquid state and a motion-restricted crystalline state, an MRI signal intensity changes by several orders of magnitude, thereby achieving an “on/off” temperature-responsive MRI effect for a polymer material.

The objective of the present disclosure is achieved through the following specific technical solutions:

A method for MRI with a non-metallic temperature-responsive composite, including the following steps:
(S1) preparation of spinning solutions: dissolving a polymer material in a solvent to obtain a polymer solution, and dissolving an organic hydrogen-containing molecular contrast agent in a solvent to obtain a contrast agent solution;
(S2) mixing the polymer solution and the contrast agent solution, and subjecting a resulting mixture to electrospinning, such that the organic hydrogen-containing molecular contrast agent is loaded in a polymer fiber;
(S3) collecting and drying a composite fiber product, and immersing the composite fiber product in water to exclude air in the fiber; and
(S4) according to a response temperature of the organic hydrogen-containing molecular contrast agent, providing a positive imaging effect under T1WI for the polymer material in the composite fiber product at a temperature higher than the response temperature of the organic hydrogen-containing molecular contrast agent; and not providing an imaging effect at a temperature lower than the response temperature of the organic hydrogen-containing molecular contrast agent.

The polymer material in step (S1) is one or a mixture of two or more selected from the group consisting of a polyester polymer and a derivative thereof, a polyolefin polymer and a derivative thereof, a polyamide (PA) polymer and a derivative thereof, a starch and a derivative thereof, cellulose and a derivative thereof, chitosan, polyoxymethylene (POM), hyaluronic acid (HA), fibrin, silk fibroin (SF), and a random copolymer and block copolymer of the above polymers.

Preferably, the polyester polymer and the derivative thereof refer to at least one selected from the group consisting of polyglycolic acid (PGA), polylactic acid (PLA), polycaprolactone (PCL), polyglycolic acid (PGA), polymethyl methacrylate (PMMA), polyethylene terephthalate (PET), polybutylene terephthalate (PBT), and polycarbonate (PC); the polyolefin polymer and the derivative thereof refer to at least one selected from the group consisting of polyethylene (PE), polypropylene (PP), polyvinyl chloride (PVC), polytetrafluoroethylene (PTFE), polyisoprene, polyvinylpyrrolidone (PVP), polyvinyl alcohol (PVA), and polyacrylonitrile (PAN); the PA polymer and the derivative thereof refer to at least one selected from the group consisting of nylon 6, nylon 66, nylon 610, and nylon 1212; the starch and the derivative thereof refer to a hydroxyethyl starch (HES) and/or a carboxymethyl starch (CMS); the cellulose and the derivative thereof refer to at least one selected from the group consisting of cellulose acetate, methylcellulose, ethylcellulose, hydroxyethyl cellulose (HEC), cyanoethyl cellulose (CEC), hydroxypropyl cellulose (HPC), and hydroxypropyl methylcellulose (HPMC); and the random copolymer and block copolymer refer to at least one selected from the group consisting of an poly (D,L-lactic acid) (PDLLA) copolymer, a polyethylene glycol (PEG)-PLA block copolymer, a PEG-PCL block copolymer, a PEG-PVP block copolymer, a polystyrene (PS)-polybutadiene (PB) block copolymer, a styrene-butadiene-styrene triblock copolymer, a PS-poly (ethylene-butylene)-PS block copolymer, a styrene-isoprene/butadiene-styrene block copolymer, and a PS-PB-PS block copolymer.

The organic hydrogen-containing molecular contrast agent in step (S1) is one or a mixture of two or more selected from the group consisting of a long-chain fatty monoacid, a long-chain fatty monoalcohol, a monoacid monoalcohol long-chain fatty ester, and a monoacid polyol long-chain fatty ester; and the response temperature is −18° C. to 70° C.

Preferably, the long-chain fatty monoacid is a fatty monoacid with 8 to 12 carbon atoms, and the response temperature is 13° C. to 70° C.; the long-chain fatty monoalcohol is a fatty monoalcohol with 8 to 18 carbon atoms, and the response temperature is −16.7° C. to 59° C.; the monoacid monoalcohol long-chain fatty ester is an ester with 16 to 28 carbon atoms produced by a long-chain fatty monoacid and a long-chain fatty monoalcohol, and the response temperature is −18° C. to 38° C.; and the monoacid polyol long-chain fatty ester is an ester compound that is produced by glycerol, sucrose, and a long-chain fatty monoacid with 8 to 14 carbon atoms, and the response temperature is 3.2° C. to 70° C.

More preferably, the fatty monoacid with 8 to 24 carbon atoms and the response temperature thereof are shown in Table 1; the fatty monoalcohol with 8 to 18 carbon atoms and the response temperature thereof are shown in Table 2; the ester with 16 to 28 carbon atoms that is produced by a long-chain fatty monoacid and a long-chain fatty monoalcohol, and the response temperature thereof are shown in Table 3; and the ester compound that is produced by glycerol, sucrose, and a long-chain fatty monoacid with 8 to 14 carbon atoms, and the response temperature thereof are shown in Table 4.

TABLE 1
Preferred fatty monoacids with 8 to 24 carbon
atoms and MRI response temperatures thereof

| | | Response temperature |
|---|---|---|
| No. | Compound | (° C.) |
| 1 | Octanoic acid (caprylic acid) | 16.0 |
| 2 | Nonanoic acid | 12.4 |
| 3 | Decanoic acid (capric acid) | 31.5 |
| 4 | Undecanoic acid | 35.0 |
| 5 | Dodecanoic acid (lauric acid) | 44.2 |
| 6 | Tridecanoic acid | 42.0 |
| 7 | Tetradecanoic acid (myristic acid) | 54.1 |
| 8 | Pentadecanoic acid | 52.0 |
| 9 | Hexadecanoic acid (palmitic acid) | 67.2 |
| 10 | Heptadecanoic acid | 60.0 |
| 11 | Octadecanoic acid (stearic acid) | 69.6 |
| 12 | cis-9-Octadecenoic acid (oleic acid) | 13.5 |
| 13 | cis-11-Octadecenoic acid (isooleic acid) | 44.0 |
| 14 | cis-13-Docosenoic acid (erucic acid) | 33.5 |
| 15 | cis-15-Tetracosenoic acid (nervonic acid) | 39.0 |

TABLE 2
Preferred fatty monoalcohols with 8 to 18 carbon
atoms and MRI response temperatures thereof

| No. | Compound | Response temperature (° C.) |
|---|---|---|
| 1 | Octanol (capryl alcohol) | −16.7 |
| 2 | Nonanol | −5.0 |
| 3 | Decanol (capric alcohol) | 6.0 |
| 4 | Undecanol | 19.0 |
| 5 | Dodecanol (lauryl alcohol) | 24.0 |
| 6 | Tridecanol | 32.0 |
| 7 | Tetradecanol (myristyl alcohol) | 36-38 |
| 8 | Pentadecanol | 43-46 |
| 9 | Hexadecanol (cetyl alcohol) | 43-46 |
| 10 | Heptadecanol | 51-55 |
| 11 | Octadecanol (stearyl alcohol) | 56-59 |

TABLE 3
Preferred ester compounds with 16 to 28 carbon atoms that each
are produced by a long-chain fatty monoacid and a long-chain
fatty monoalcohol, and MRI response temperatures thereof

| No. | Compound | Response temperature (° C.) |
|---|---|---|
| 1 | Octyl caprylate | −18.0 |
| 2 | Decyl decanoate | 9.7 |
| 3 | Ethyl stearate | 34-38 |
| 4 | Lauryl laurate | 29-31 |
| 5 | Myristyl myristate | 36.2 |

TABLE 4
Preferred ester compounds that each are produced by glycerol,
sucrose, and a long-chain fatty monoacid with 8 to 14
carbon atoms, and MRI response temperatures thereof

| No. | Compound | Response temperature (° C.) |
|---|---|---|
| 1 | Glycerol trimyristate | 56-57 |
| 2 | Glycerol monooleate | 38.5 |
| 3 | Glycerol dioleate | 3.2-3.8 |
| 4 | Glycerol monolaurate | 63.0 |
| 5 | Sucrose trioctanoate | 50-70 |

The solvent in step (S1) is one or a mixture of two or more selected from the group consisting of pentane, n-hexane, methylcyclohexane (MCH), dichloromethane (DCM), trichloromethane (TCM), dichloroethane (DCE), tetrachloroethane, carbon tetrachloride (CTC), methyl acrylate (MA), tetrahydrofuran (THF), methyltetrahydrofuran (MTHF), N,N-dimethylformamide (DMF), N,N-dimethylacetamide (DMA), dimethyl sulfoxide (DMSO), diethyl ether, petroleum ether, acetone, formic acid, acetic acid, trifluoroacetic acid (TFA), hexafluoroisopropanol (HFIP), xylene, toluene, phenol, chlorobenzene, nitrobenzene, cresol, anisole, methanol, ethanol, 1-propanol, 2-propanol, 1-butanol, 2-butanol, and pentanol.

In step (S1), the polymer solution has a concentration of 5 wt % to 60 wt %, and the contrast agent solution has a concentration of 20 wt % to 90 wt %.

The electrospinning in step (S2) is conducted under the following conditions: a liquid supply rate of a liquid supply device: 0.1 mL/h to 10 mL/h; a distance between a spinning nozzle and a collection device: 5 cm to 50 cm; a high voltage at the spinning nozzle: 10 kV to 50 kV; and a high voltage at the collection device: 0 kV to −50 kV.

In step (S3), the air in the fiber is excluded due to the following reason: The magnetic susceptibility of the air is significantly different from the magnetic susceptibility of the polymer composite fiber, which affects the inhomogeneity of a magnetic field and thus causes a poor MRI imaging effect. Therefore, there cannot be air residue.

In step (S3), the final composite fiber product provides a positive imaging effect under T1WI for the polymer material at a temperature higher than the response temperature of the organic hydrogen-containing molecular contrast agent; and does not provide an imaging effect at a temperature lower than the response temperature of the organic hydrogen-containing molecular contrast agent with a signal of the material itself not affected.

A composite fiber prepared by the method is provided.

The composite fiber prepared by the above process can be used: (1) to provide a temperature-responsive MRI signal for a polymer material, such as a polymer tissue engineering scaffold; (2) as a temperature calibration standard in MRI; (3) to determine a temperature distribution of an environment in which a composite fiber is located.

Compared with the prior art, the preparation method and the product thereof in the present disclosure have the following advantages and beneficial effects:
(1) The composite fiber prepared by the method of the present disclosure does not include a metal and does not produce an additional magnetic field to an environment. The acids, the alcohols, and the ester compounds including the same are not toxic or exhibit low toxicity to the human body.
(2) The composite fiber product provides a positive imaging effect under T1WI for the polymer material at a temperature higher than the response temperature of the organic hydrogen-containing molecular contrast agent; and does not provide an imaging effect at a temperature lower than the response temperature of the organic hydrogen-containing molecular contrast agent with a signal of the material itself not affected, thereby achieving an “on/off” imaging effect.

### BRIEF DESCRIPTION OF THE DRAWINGS

FIG. 1 shows a relationship between a correlation time of a molecule (Tc) and relaxation times.

FIG. 2 is a schematic diagram of an electrospinning device.

FIG. 3 shows variable-temperature MRI results of Example 1 and Comparative Example 1.

FIG. 4 shows the test results of Comparative Example 1 by a variable-temperature low-field nuclear magnetic resonance (NMR) spectrometer.

FIG. 5 shows variable-temperature MRI results of Example 2.

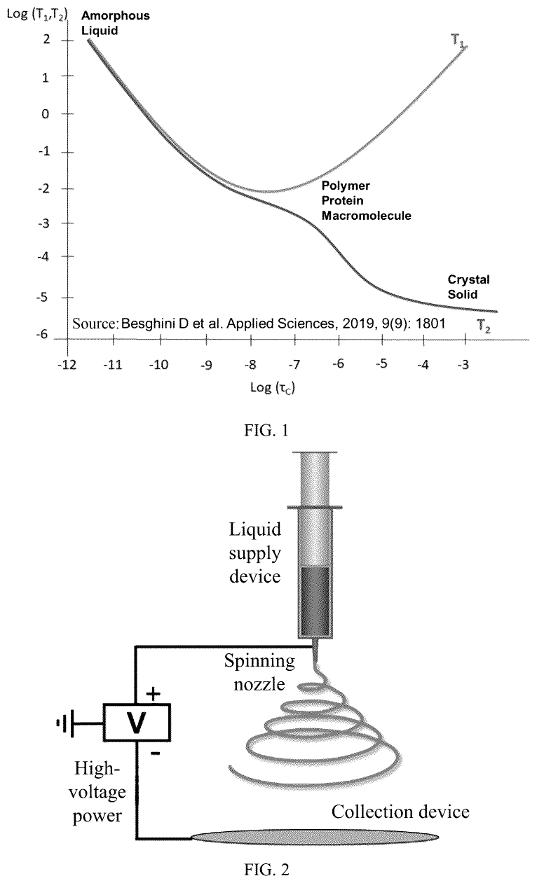

### DETAILED DESCRIPTION OF THE EMBODIMENTS

The present disclosure will be described in further detail below with reference to examples and accompanying drawings, but the implementations of the present disclosure are not limited thereto.

### Example 1

PBS was dissolved in TCM to obtain a polymer solution with a mass concentration of 60%, and lauric acid was dissolved in DCM to obtain a contrast agent solution with a mass concentration of 75%; and the polymer solution and the contrast agent solution were mixed in a mass ratio of 1:2 to obtain a spinning solution.

The spinning solution was subjected to electrospinning under the following parameters: a liquid supply rate of a liquid supply device: 3 mL/h; a distance between a spinning nozzle and a collection device: 20 cm; a high voltage at the spinning nozzle: 20 kV; and a high voltage at the collection device: −1 kV.

A composite fiber product was collected, dried, and immersed in water to exclude air in the fiber.

A response temperature of the fiber was 44.2° C., which was determined by a differential scanning calorimeter (DSC).

### Example 2

PET was dissolved in TCM to obtain a polymer solution with a mass concentration of 45%, and lauryl alcohol was dissolved in DCM to obtain a contrast agent solution with a mass concentration of 75%; and the polymer solution and the contrast agent solution were mixed in a mass ratio of 1:2 to obtain a spinning solution.

The spinning solution was subjected to electrospinning under the following parameters: a liquid supply rate of a liquid supply device: 3 mL/h; a distance between a spinning nozzle and a collection device: 20 cm; a high voltage at the spinning nozzle: 20 kV; and a high voltage at the collection device: −1 kV.

A composite fiber product was collected, dried, and immersed in water to exclude air in the fiber.

A response temperature of the fiber was 24.0° C., which was determined by DSC.

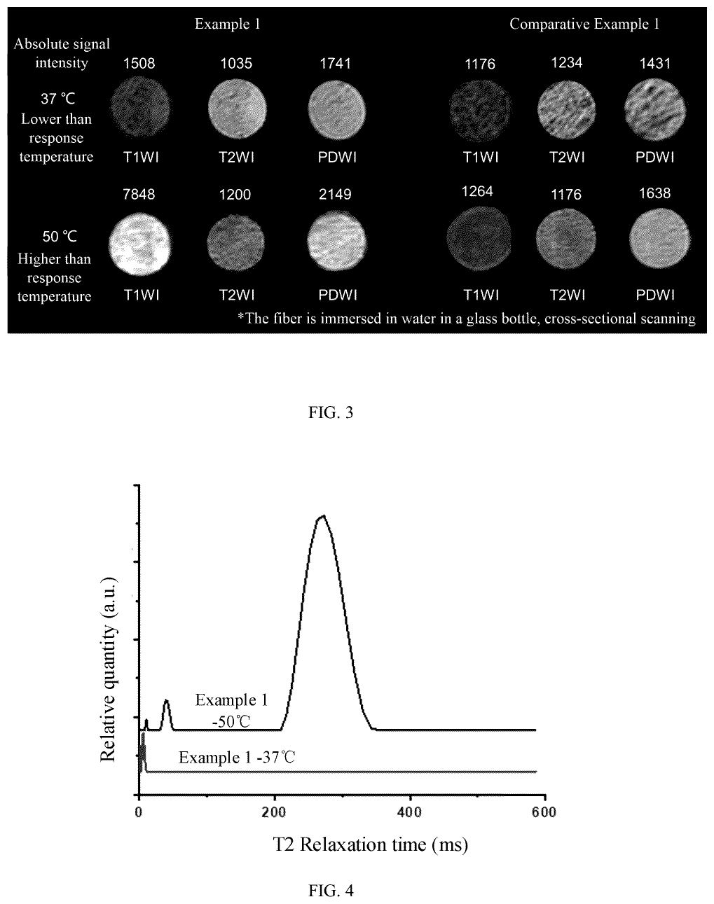

To illustrate that an MRI signal provided by the organic hydrogen-containing molecular contrast agent used in the present disclosure came from an aliphatic chain in the contrast agent, rather than other groups, the products of Examples 1 and 2 each were subjected to variable-temperature MRI (as shown in FIG. 3 and FIG. 5). It can be seen from MRI results of Examples 1 and 2 that lauric acid and lauryl alcohol can provide a positive imaging effect under T1WI for the polymer fiber at a temperature higher than the response temperature, and does not provide an imaging effect at a temperature lower than the response temperature; and the two products have no imaging effect for T2WI and PDWI sequences, indicating that lauric acid has a similar imaging effect to lauryl alcohol, and hydrogen atoms affecting their imaging abilities are provided by long-chain fats.

### Example 3

PVA was dissolved in water to obtain a polymer solution with a mass concentration of 5%, and glycerol trimyristate was dissolved in THF to obtain a contrast agent solution with a mass concentration of 60%; and the polymer solution and the contrast agent solution were mixed in a mass ratio of 1:2 to obtain a spinning solution.

The spinning solution was subjected to electrospinning under the following parameters: a liquid supply rate of a liquid supply device: 0.1 mL/h; a distance between a spinning nozzle and a collection device: 50 cm; a high voltage at the spinning nozzle: 50 kV; and a high voltage at the collection device: −1 kV.

A composite fiber product was collected, dried, and immersed in water to exclude air in the fiber.

A response temperature of the fiber was 56.2° C., which was determined by DSC.

### Example 4

A styrene-butadiene-styrene block copolymer was dissolved in THF to obtain a polymer solution with a mass concentration of 40%, and isooleic acid was dissolved in acetone to obtain a contrast agent solution with a mass concentration of 30%; and the polymer solution and the contrast agent solution were mixed in a mass ratio of 2:3 to obtain a spinning solution.

The spinning solution was subjected to electrospinning under the following parameters: a liquid supply rate of a liquid supply device: 5 mL/h; a distance between a spinning nozzle and a collection device: 30 cm; a high voltage at the spinning nozzle: 40 kV; and a high voltage at the collection device: −20 kV.

A composite fiber product was collected, dried, and immersed in water to exclude air in the fiber.

A response temperature of the fiber was 44.0° C., which was determined by DSC.

### Example 5

An PDLLA random copolymer was dissolved in DCM to obtain a polymer solution with a mass concentration of 24%, and myristyl myristate was dissolved in DMA to obtain a contrast agent solution with a mass concentration of 90%; and the polymer solution and the contrast agent solution were mixed in a mass ratio of 1:1 to obtain a spinning solution.

The spinning solution was subjected to electrospinning under the following parameters: a liquid supply rate of a liquid supply device: 5 mL/h; a distance between a spinning nozzle and a collection device: 30 cm; a high voltage at the spinning nozzle: 10 kV; and a high voltage at the collection device: −50 kV.

A composite fiber product was collected, dried, and immersed in water to exclude air in the fiber.

A response temperature of the fiber was 36.2° C., which was determined by DSC.

### Example 6

Cellulose acetate was dissolved in a mixed solvent of DMA and acetone in a ratio of 2 wt. %: 1 wt. % to obtain a polymer solution with a mass concentration of 10%, and glycerol monooleate was dissolved in diethyl ether to obtain a contrast agent solution with a mass concentration of 20%; and the polymer solution and the contrast agent solution were mixed in a mass ratio of 1:1 to obtain a spinning solution.

The spinning solution was subjected to electrospinning under the following parameters: a liquid supply rate of a liquid supply device: 10 mL/h; a distance between a spinning nozzle and a collection device: 25 cm; a high voltage at the spinning nozzle: 25 kV; and a high voltage at the collection device: −10 kV.

A composite fiber product was collected, dried, and immersed in water to exclude air in the fiber.

A response temperature of the fiber was 38.5° C., which was determined by DSC.

### Example 7

Nylon 1212 was dissolved in formic acid to obtain a polymer solution with a mass concentration of 20%, and glycerol monolaurate was dissolved in diethyl ether to obtain a contrast agent solution with a mass concentration of 90%; and the polymer solution and the contrast agent solution were mixed in a mass ratio of 1:2 to obtain a spinning solution.

The spinning solution was subjected to electrospinning under the following parameters: a liquid supply rate of a liquid supply device: 10 mL/h; a distance between a spinning nozzle and a collection device: 5 cm; a high voltage at the spinning nozzle: 10 kV; and a high voltage at the collection device: −40 kV.

A composite fiber product was collected, dried, and immersed in water to exclude air in the fiber.

A response temperature of the fiber was 63.0° C., which was determined by DSC.

### Comparative Example 1

PBS was dissolved in TCM to obtain a polymer solution with a mass concentration of 20%.

The spinning solution was subjected to electrospinning under the following parameters: a liquid supply rate of a liquid supply device: 3 mL/h; a distance between a spinning nozzle and a collection device: 20 cm; a high voltage at the spinning nozzle: 20 kV; and a high voltage at the collection device: −1 kV.

A composite fiber product was collected, dried, and immersed in water to exclude air in the fiber.

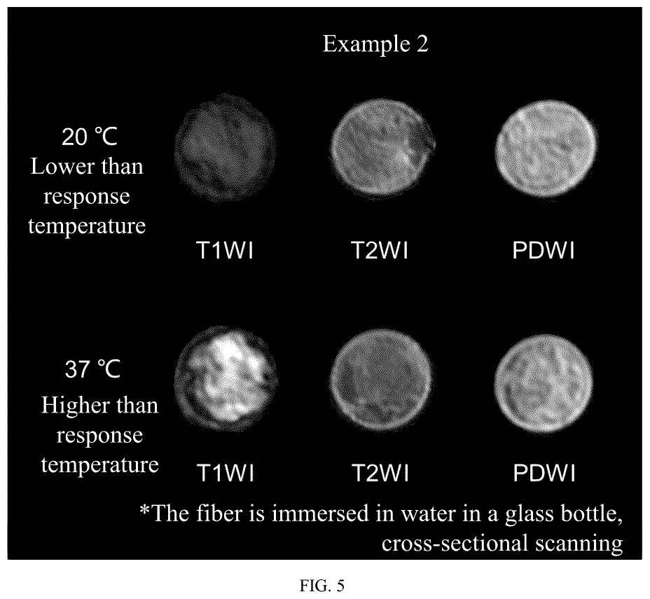

To illustrate the effectiveness of the method of the present disclosure, the products obtained in Example 1 and Comparative Example 1 each was subjected to variable-temperature MRI (as shown in FIG. 3), and the product of Example 1 was tested by a variable-temperature low-field NMR spectrometer to determine a relaxation time of lauric acid (as shown in FIG. 4).

As shown in FIG. 3, the products of Example 1 and Comparative Example 1 each were scanned under T1WI, T2WI, and PDWI sequences at 50° C. (higher than the response temperature of lauric acid) and 37° C. (lower than the response temperature of lauric acid), where a signal intensity of the T1WI sequence mainly reflected a T1 relaxation time, and was secondarily controlled by a proton density and a T2 relaxation time; a signal intensity of the T2WI sequence mainly reflected a T2 relaxation time, and was secondarily controlled by a proton density and a T1 relaxation time; and a signal intensity of the PDWI sequence mainly reflected a proton density, and was secondarily controlled by relaxation times T1 and T2.

It can be seen from FIG. 3 that the product of Example 1 has a high T1WI signal intensity at 50° C., and does not have a high signal intensity at 37° C.; and the product of Comparative Example 1 does not show a similar situation, indicating that the organic hydrogen-containing molecular contrast agent loaded in the composite fiber has prominent temperature responsiveness.

In addition, since PDWI mainly reflected a proton density, it could be used as a correction benchmark to compare relative signal intensities of single hydrogen atoms in different states. It can be seen from imaging results of Comparative Example 1 that a ratio of a T1WI sequence intensity to a PDWI sequence intensity is close to a ratio of a T2WI sequence intensity to a PDWI sequence intensity at different temperatures, indicating that signal intensities provided by single hydrogen atoms are similar. A ratio of a T1WI sequence intensity to a PDWI sequence intensity and a ratio of a T2WI sequence intensity to a PDWI sequence intensity in Example 1 are close to that in Comparative Example 1 at 37° C., indicating that, at this temperature, hydrogen atoms providing a signal come from water molecules infiltrating the fiber. In Example 1, a ratio of T1WI to PDWI at 50° C. was much greater than a ratio of T1WI to PDWI at 37° C., indicating that there are hydrogen atoms with a high-intensity signal (from lauric acid) in the system.

It can be seen from FIG. 4 that a T2 relaxation time of lauric acid in the composite fiber is mainly about 280 ms at a temperature higher than the response temperature; and at a temperature lower than the response temperature, the T2 relaxation time is about 5 ms, which is decreased by about 2 orders of magnitude.

In addition, at a temperature higher than the response temperature, a T1 relaxation time of lauric acid in the composite fiber determined by a low-field NMR spectrometer is to be approximately 278 ms; and at a temperature lower than the response temperature, because T2 relaxation time is too short, T1 relaxation time cannot be characterized. However, the effectiveness of the method of the present disclosure may be illustrated by the formula (1). For the composite, the proton density of lauric acid remains unchanged before and after a temperature change, that is, N_{(H)i} remains unchanged and is assumed to be 1; and the T1 relaxation time of lauric acid is unmeasurable at a temperature lower than the response temperature. However, it is not difficult to see that, since TR and T1 both are greater than 0 and

$$( 1 - {e}^{- \frac{TR}{T 1}} )$$

is always less than 1, the maximum value of 1 is taken for estimation. With TE=105 ms for the T2WI sequence as an example, signal intensities of lauric acid in the sample of Example 1 at the two temperatures are as follows:

$${S}_{50 ° C .} ∝ 1 \cdot 1 \cdot {e}^{- \frac{105 ms}{280 ms}} = 0.687$$

$${S}_{37 ° C .} ∝ 1 \cdot 1 \cdot {e}^{- \frac{105 ms}{5 ms}} = 7.58 * {10}^{- 10} .$$

Signal values of the two differ by nearly 9 orders of magnitude, and thus lauric acid does not provide an MRI signal at a temperature lower than the response temperature.

In summary, data from Example 1 and Comparative Example 1 shows that the composite fiber of Example 1 has temperature responsiveness; lauric acid can provide a T1WI positive imaging effect for the polymer fiber; and at a temperature lower than the response temperature, a signal in the system comes from water molecules infiltrating the fiber, rather than lauric acid molecules, which achieves the purpose of providing an “on/off” MRI effect.

1. A method for magnetic resonance imaging (MRI) with a non-metallic temperature-responsive composite, comprising the following steps:
(S1) preparation of spinning solutions: dissolving a polymer material in a solvent to obtain a polymer solution, and dissolving an organic hydrogen-containing molecular contrast agent in a solvent to obtain a contrast agent solution;
(S2) mixing the polymer solution and the contrast agent solution, and subjecting a resulting mixture to electrospinning, such that the organic hydrogen-containing molecular contrast agent is loaded in a polymer fiber;
(S3) collecting and drying a composite fiber product, and immersing the composite fiber product in water to exhaust air in the fiber; and
(S4) according to a response temperature of the organic hydrogen-containing molecular contrast agent, providing a positive imaging effect under T1-weighted imaging (T1WI) for the polymer material in the composite fiber product at a temperature higher than the response temperature of the organic hydrogen-containing molecular contrast agent; and not providing an imaging effect at a temperature lower than the response temperature of the organic hydrogen-containing molecular contrast agent.

2. The method according to claim 1, wherein the polymer material in step (S1) is one or a mixture of two or more selected from the group consisting of a polyester polymer and a derivative thereof, a polyolefin polymer and a derivative thereof, a polyamide (PA) polymer and a derivative thereof, a starch and a derivative thereof, cellulose and a derivative thereof, chitosan, polyoxymethylene (POM), hyaluronic acid (HA), fibrin, silk fibroin (SF), and a random copolymer and a block copolymer of the above polymers.

3. The method according to claim 2, wherein
the polyester polymer and the derivative thereof refer to at least one selected from the group consisting of polyglycolic acid (PGA), polylactic acid (PLA), polycaprolactone (PCL), polyglycolic acid (PGA), polymethyl methacrylate (PMMA), polyethylene terephthalate (PET), polybutylene terephthalate (PBT), and polycarbonate (PC); the polyolefin polymer and the derivative thereof refer to at least one selected from the group consisting of polyethylene (PE), polypropylene (PP), polyvinyl chloride (PVC), polytetrafluoroethylene (PTFE), polyisoprene, polyvinylpyrrolidone (PVP), polyvinyl alcohol (PVA), and polyacrylonitrile (PAN); the PA polymer and the derivative thereof refer to at least one selected from the group consisting of nylon 6, nylon 66, nylon 610, and nylon 1212; the starch and the derivative thereof refer to a hydroxyethyl starch (HES) and/or a carboxymethyl starch (CMS); the cellulose and the derivative thereof refer to at least one selected from the group consisting of cellulose acetate, methylcellulose, ethylcellulose, hydroxyethyl cellulose (HEC), cyanoethyl cellulose (CEC), hydroxypropyl cellulose (HPC), and hydroxypropyl methylcellulose (HPMC); and the random copolymer and the block copolymer refer to at least one selected from the group consisting of an poly (D,L-lactic acid) (PDLLA) copolymer, a polyethylene glycol (PEG)-PLA block copolymer, a PEG-PCL block copolymer, a PEG-PVP block copolymer, a polystyrene (PS)-polybutadiene (PB) block copolymer, a styrene-butadiene-styrene triblock copolymer, a PS-poly (ethylene-butylene)-PS block copolymer, a styrene-isoprene/butadiene-styrene block copolymer, and a PS-PB-PS block copolymer.

4. The method according to claim 1, wherein the organic hydrogen-containing molecular contrast agent in step (S1) is one or a mixture of two or more selected from the group consisting of a long-chain fatty monoacid, a long-chain fatty monoalcohol, a monoacid monoalcohol long-chain fatty ester, and a monoacid polyol long-chain fatty ester; and the response temperature is −18° C. to 70° C.

5. The method according to claim 4, wherein
the long-chain fatty monoacid is a fatty monoacid with 8 to 12 carbon atoms, and the response temperature is 13° C. to 70° C.; the long-chain fatty monoalcohol is a fatty monoalcohol with 8 to 18 carbon atoms, and the response temperature is −16.7° C. to 59° C.; the monoacid monoalcohol long-chain fatty ester is an ester with 16 to 28 carbon atoms produced by a long-chain fatty monoacid and a long-chain fatty monoalcohol, and the response temperature is −18° C. to 38° C.; and the monoacid polyol long-chain fatty ester is an ester compound that is produced by glycerol, sucrose, and a long-chain fatty monoacid with 8 to 14 carbon atoms, and the response temperature is 3.2° C. to 70° C.

6. The method according to claim 1, wherein the solvent in step (S1) is one or a mixture of two or more selected from the group consisting of pentane, n-hexane, methylcyclohexane (MCH), dichloromethane (DCM), trichloromethane (TCM), dichloroethane (DCE), tetrachloroethane, carbon tetrachloride (CTC), methyl acrylate (MA), tetrahydrofuran (THF), methyltetrahydrofuran (MTHF), N,N-dimethylformamide (DMF), N,N-dimethylacetamide (DMA), dimethyl sulfoxide (DMSO), diethyl ether, petroleum ether, acetone, formic acid, acetic acid, trifluoroacetic acid (TFA), hexafluoroisopropanol (HFIP), xylene, toluene, phenol, chlorobenzene, nitrobenzene, cresol, anisole, methanol, ethanol, 1-propanol, 2-propanol, 1-butanol, 2-butanol, and pentanol.

7. The method according to claim 1, wherein in step (S1), the polymer solution has a concentration of 5 wt % to 60 wt %, and the contrast agent solution has a concentration of 20 wt % to 90 wt %.

8. The method according to claim 1, wherein the electrospinning in step (S2) is conducted under the following conditions: a liquid supply rate of a liquid supply device: 0.1 mL/h to 10 mL/h; a distance between a spinning nozzle and a collection device: 5 cm to 50 cm; a high voltage at the spinning nozzle: 10 kV to 50 kV; and a high voltage at the collection device: 0 kV to −50 kV.

9. A non-metallic temperature-responsive composite for MRI prepared by steps (S1) to (S3) of the method according to any one of claims 1 to 8.

10. A use of the non-metallic temperature-responsive composite for MRI according to claim 9: (1) to provide a temperature-responsive MRI signal for a polymer material; (2) as a temperature calibration standard in MRI; and (3) to determine a temperature distribution of an environment in which a composite fiber is located.
